(19) United States
(12) Patent Application Publication
(10) Pub. No.: US 2026/0118331 A1
(43) Pub. Date: Apr. 30, 2026

(54) INTELLIGENT VOLATILE ORGANIC COMPOUND SEQUESTERING

(71) Applicant: International Business Machines Corporation, Armonk, NY (US)

(72) Inventors: Rebecca N. Morones, Berthoud, CO (US); John S. Werner, Fishkill, NY (US); Arkadiy O. Tsfasman, Wappingers Falls, NY (US); Christopher V. DeRobertis, Hopewell Junction, NY (US)

(21) Appl. No.: 18/926,451

(22) Filed: Oct. 25, 2024

(51) Int. Cl. G01N 33/00

(52) U.S. Cl. CPC G01N 33/0047; G01N 33/0031

(57) ABSTRACT

A computer-implemented method that reduces volatile organic compounds in an atmosphere is provided. A number of processor units extract a physical layout of a data center from a database. A number of volatile organic compound sensors captures a set of volatile organic compound data within the data center. Responsive to the capturing, the number of processor units creates a volatile organic compound map based on the physical layout and the set of volatile organic compound data. The number of processor units reposition a mobile volatile organic compound sequestering device to one or more locations in the data center based on the volatile organic compound map and the physical layout. According to other illustrative embodiments, a computer system and computer program product for reducing volatile organic compounds in an atmosphere are provided.

### BACKGROUND

The disclosure relates generally to controlling indoor pollutants and more specifically to sequestering volatile organic compounds in data centers.

Volatile organic compounds (VOCs) are off-gassed from and/or emitted by electrical hardware and/or electronics. This can be especially bad on new products. These VOCs can cause irritation to the eyes/nose/throat, birth defects, cancer, and have other harmful effects. This issue is compounded in a data center environment which can contain hundreds or thousands of systems each of which increases the risk of the harmful effects.

Chemical filters have been incorporated into the hot air gas exit port of servers to trap VOCs emanating from within the server. For example, chemical filters remove volatile organic compounds by capturing and neutralizing harmful compounds. The use of these filters improve air quality and protect equipment. Chemical filters are composed of activated carbon or other adsorbent materials. These filters utilize the process of adsorption where VOC molecules adhere to the surface of the filter material. This filtering prevents VOCs from entering the surrounding environment and reduces the risk of corrosion to sensitive electronic components and enhances overall air quality. Additionally, some filters may include catalytic elements that chemically break down VOCs into less harmful substances.

### SUMMARY

According to one illustrative embodiment, a computer-implemented method for reducing volatile organic compounds in an atmosphere is provided. A number of processor units extract a physical layout of a data center from a database. A number of volatile organic compound sensors captures a set of volatile organic compound data within the data center. Responsive to the capturing, the number of processor units creates a volatile organic compound map based on the physical layout and the set of volatile organic compound data. The number of processor units reposition a mobile volatile organic compound sequestering device to one or more locations in the data center based on the volatile organic compound map and the physical layout. According to other illustrative embodiments, a computer system and computer program product for reducing volatile organic compounds in an atmosphere are provided.

Embodiments of this disclosure include systems and methods to reduce VOCs and/or toxic gases in a data center that a user/worker could be exposed to. The system creates a VOC mapping, a toxic gas mapping and/or an airflow mapping over time (that can be stored in database holding multiple profiles at different days/times) within the data center which is constantly changing based on workload, new system installation, system removals, etc. A sequestering machine including a sequestering device can be positioned to the point in the data center where most of the VOCs and/or toxic gasses would pass through to better protect users in the data center. The sequestering device can be located within a robot, or just a device that is manually moved by a data center worker, repositioned to a position of high airflow where it is likely to capture the most VOCs. Positioning could be predictive based on the mapping stored in the database. The sequestering device can contain multiple VOC filters as well as an optional suction device (e.g., vacuum). The sequestering device can also be positioned behind newly installed systems which off-gas/emit relatively more VOCs and/or toxic gases during their initial warm-up and operation. During this process for new systems, the data center cooling can in addition be adjusted such that new systems temporarily run relatively hotter so that they off-gas more VOCs. Adjustments can be implemented by changing computer room air conditioner (CRAC) unit cooling or HVAC output, adjusting fan speeds of nearby systems, adjusting positioning of floor tiles, or percentage opening of a perf floor tile.

### BRIEF DESCRIPTION OF THE DRAWINGS

FIG. 1 is a block diagram of a computing environment in accordance with an illustrative embodiment;

FIG. 2 is a block diagram of a data center equipped for intelligent volatile organic compound capturing in accordance with an illustrative embodiment;

FIG. 3 is a dataflow for intelligent volatile organic compound capturing in a data center in accordance with an illustrative embodiment;

FIG. 4 is a flowchart of a process for intelligent volatile organic compound capturing in a data center in accordance with an illustrative embodiment;

FIG. 5 is a flow chart of a further process in accordance with an illustrative embodiment; and

FIG. 6 is a block diagram of a data processing system in accordance with an illustrative embodiment.

### DETAILED DESCRIPTION

Embodiments include a computer implemented method including: extracting, by a number of processor units, a physical layout of a data center from a database; capturing, by a number of volatile organic compound sensors, a set of volatile organic compound data within the data center; responsive to the capturing, creating, by the number of processor units, a volatile organic compound map based on the physical layout and the set of volatile organic compound data; and repositioning a mobile volatile organic compound sequestering device to one or more locations in the data center based on the volatile organic compound map and the physical layout. As a result, these illustrative embodiments provide a technical effect of sequestering volatile organic compounds by repositioning a mobile volatile organic compound sequestering device based on the volatile organic compound map and the physical layout.

In some embodiments, capturing, by the number of volatile organic compound sensors, the set of volatile organic compound data within the data center is performed at regular intervals. As a result, these illustrative embodiments provide a technical effect of capturing data at regular intervals.

In some embodiments, capturing is performed responsive to detecting an unexpected change in activity level on one or more systems in the data center. As a result, these illustrative embodiments provide a technical effect of detecting an unexpected change in activity level on one or more systems in the data center.

In some embodiments, repositioning comprises repositioning to at least one of where a volatile organic compound is highest above its threshold or highest as a percentage of its threshold. As a result, these illustrative embodiments provide a technical effect of sequestering volatile organic compounds by repositioning to at least one of where a volatile organic compound is highest and where a volatile organic compound exceeds a threshold level.

In some embodiments, repositioning comprises following a user moving within the data center. As a result, these illustrative embodiments provide a technical effect of sequestering volatile organic compounds based on following a user moving within the data center.

In some embodiments, repositioning is based on at least one of historical power consumption and historical workload. As a result, these illustrative embodiments provide a technical effect of sequestering volatile organic compounds based on at least one of historical power consumption and historical workload.

Some embodiments further include extracting, by the number of processor units, an activity level from each of a plurality of systems located in the data center; correlating, by the number of processor units, the activity level with the volatile organic compound map; and predicting, by the number of processor units, a future volatile organic compound map using an off-gassing decay factor. As a result, these illustrative embodiments provide a technical effect of sequestering volatile organic compounds based on extracting, by the number of processor units, an activity level from each of a plurality of systems located in the data center; correlating, by the number of processor units, the activity level with the volatile organic compound map; and predicting a future volatile organic compound map using an off-gassing decay factor. Of note, there can be other systems running in the data center.

In some embodiments, the activity level includes at least one of power consumption and workload from each of the plurality of systems located in the data center. As a result, these illustrative embodiments provide a technical effect of sequestering volatile organic compounds based on at least one of power consumption and workload from each of the plurality of systems located in the data center. Of note, there can be other systems running in the data center.

Some embodiments further include, responsive to detecting a new system in the data center, repositioning the mobile volatile organic compound sequestering device proximate the new system. As a result, these illustrative embodiments provide a technical effect of repositioning the mobile volatile organic compound sequestering device proximate the new system.

Some embodiments further include, responsive to repositioning the mobile volatile organic compound sequestering device proximate a new system, adjusting a cooling system associated with the new system in the data center to run the new system at a higher temperature than the plurality of systems to increase off-gassing of volatile organic compounds from the new system. As a result, these illustrative embodiments provide a technical effect of adjusting a cooling system associated with the new system in the data center to run the new system at a higher temperature than the plurality of systems to increase off-gassing of volatile organic compounds from the new system. Of note, there can be other systems running in the data center.

Some embodiments further include capturing, by a number of airflow sensors, airflow data within the data center. As a result, these illustrative embodiments provide a technical effect of capturing, by a number of airflow sensors, airflow data within the data center.

Some embodiments further include, responsive to a volatile organic compound threshold being exceeded, at least one of activating an audible alarm, activating a visual alarm, and locking one or more doors of the data center. As a result, these illustrative embodiments provide a technical effect of at least one of activating an audible alarm, activating a visual alarm, and locking one or more doors of the data center.

Embodiments include a computer implemented method including: extracting, by a number of processor units, a physical layout of a data center from a data center infrastructure management tool; capturing, by a number of toxic gas sensors, a set of toxic gas data within the data center; responsive to the capturing, creating, by the number of processor units, a toxic gas map based on the physical layout and the set of toxic gas data; and repositioning a mobile toxic gas sequestering device in one or more locations in the data center based on the toxic gas map and the physical layout. As a result, these illustrative embodiments provide a technical effect of sequestering toxic gases by repositioning a mobile toxic gas sequestering device based on the toxic gas map and the physical layout.

Some embodiments further include extracting, by the number of processor units, an activity level from each of a plurality of systems located in the data center; and correlating, by the number of processor units, the activity level with the toxic gas map. As a result, these illustrative embodiments provide a technical effect of extracting, by the number of processor units, an activity level from each of a plurality of systems located in the data center; and correlating, by the number of processor units, the activity level with the toxic gas map. Of note, there can be other systems running in the data center.

Some embodiments further include capturing, by a number of airflow sensors, airflow data within the data center. As a result, these illustrative embodiments provide a technical effect of capturing, by a number of airflow sensors, airflow data within the data center.

Some embodiments further include, responsive to a toxic gas threshold being exceeded, at least one of activating an audible alarm, activating a visual alarm, and locking one or more doors of the data center. As a result, these illustrative embodiments provide a technical effect of, responsive to a toxic gas threshold being exceeded, at least one of activating an audible alarm, activating a visual alarm, and locking one or more doors of the data center.

Embodiments include a computer implemented method including: extracting, by a number of processor units, a physical layout of a data center from a data center infrastructure management tool; capturing, by a number of airflow sensors, a set of airflow data within the data center; responsive to the capturing, creating, by the number of processor units, an airflow map based the physical layout and the set of airflow data; and repositioning a mobile sequestering device to one or more locations in the data center based on the airflow map and the physical layout. As a result, these illustrative embodiments provide a technical effect of repositioning the mobile sequestering device based on the airflow map and the physical layout.

Some embodiments further include, responsive to repositioning the mobile sequestering device proximate a new system, adjusting a cooling system associated with the new system in the data center to run the new system at a higher temperature than the plurality of systems to increase off-gassing of volatile organic compounds from the new system. As a result, these illustrative embodiments provide a technical effect of adjusting a cooling system associated with the new system in the data center to run the new system at a higher temperature than the plurality of systems to increase off-gassing of volatile organic compounds from the new system. Of note, there can be other systems running in the data center.

Some embodiments further include capturing, by a number of airflow sensors, airflow data within the data center. As a result, these illustrative embodiments provide a technical effect of capturing airflow data within the data center.

Some embodiments further include extracting, by the number of processor units, an activity level from each of a plurality of systems located in the data center; and correlating, by the number of processor units, the activity level with the airflow map. As a result, these illustrative embodiments provide a technical effect of extracting, by the number of processor units, an activity level from each of a plurality of systems located in the data center; and correlating, by the number of processor units, the activity level with the airflow map. Of note, there can be other systems running in the data center.

Embodiments include a computer system including: a processor set; a set of one or more computer readable storage media; program instructions, collectively stored in the set of one or more storage media, for causing the processor set to perform the following computer operations: extract, by a number of processor units, a physical layout of a data center from a database; capture, by a number of volatile organic compound sensors, a set of volatile organic compound data within the data center; responsive to the capture, create, by the number of processor units, a volatile organic compound map based on the physical layout and the set of volatile organic compound data; and reposition a mobile volatile organic compound sequestering device to one or more locations in the data center based on the volatile organic compound map and the physical layout. As a result, these illustrative embodiments provide a technical effect of sequestering volatile organic compounds by repositioning a mobile volatile organic compound sequestering device using the computer system.

In some embodiments the program instructions cause the processor set to perform the following computer operations: extract, by the number of processor units, an activity level from each of a plurality of systems located in the data center; and correlate, by the number of processor units, the activity level with the volatile organic compound map. As a result, these illustrative embodiments provide a technical effect of extracting, by the number of processor units, an activity level from each of a plurality of systems located in the data center; and correlating, by the number of processor units, the activity level with the volatile organic compound map. Of note, there can be other systems running in the data center.

In some embodiments the program instructions cause the processor set to perform the following computer operations: capture, by a number of airflow sensors, airflow data within the data center. As a result, these illustrative embodiments provide a technical effect of capturing, by a number of airflow sensors, airflow data within the data center.

In some embodiments the program instructions cause the processor set to perform at least one of the following computer operations, responsive to a volatile organic compound threshold being exceeded, activate an audible alarm, activate a visual alarm, and lock one or more doors of the data center. As a result, these illustrative embodiments provide a technical effect of activating an audible alarm, activating a visual alarm, and locking one or more doors of the data center.

Embodiments include a computer program product including a set of one or more computer-readable storage media; program instructions, collectively stored in the set of one or more storage media, for causing a processor set to perform the following computer operations: extract, by a number of processor units, a physical layout of a data center from a database; capture, by a number of volatile organic compound sensors, a set of volatile organic compound data within the data center; responsive to the capture, create, by the number of processor units, a volatile organic compound map based on the physical layout and the set of volatile organic compound data; and reposition a mobile volatile organic compound sequestering device to one or more locations in the data center based on the volatile organic compound map and the physical layout. As a result, these illustrative embodiments provide a technical effect of sequestering volatile organic compounds by repositioning a mobile volatile organic compound sequestering device using the computer program product.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

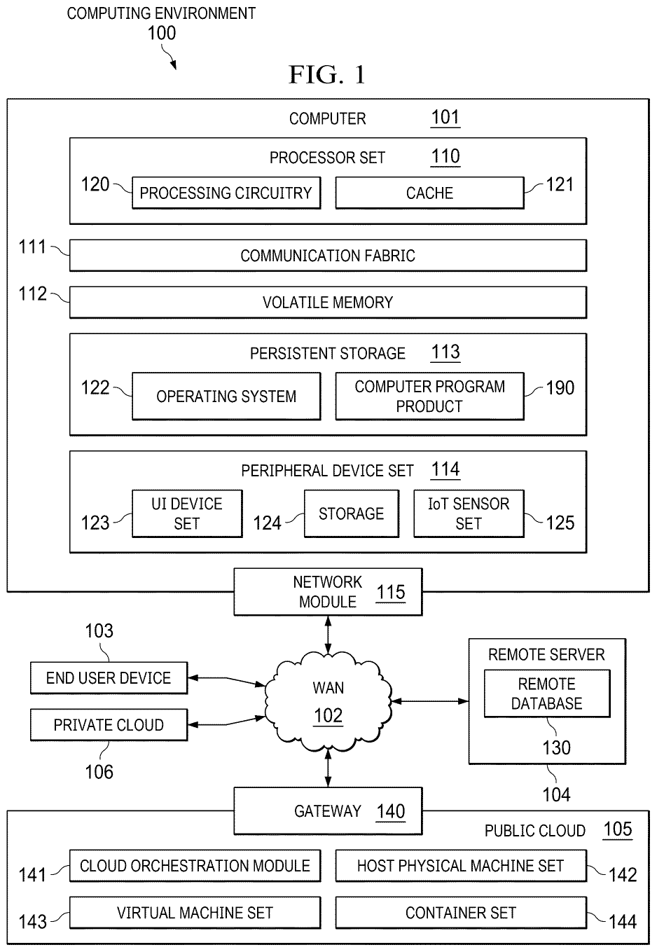

With reference now to the figures in particular with reference to FIG. 1, a block diagram of a computing environment is depicted in accordance with an illustrative embodiment. Computing environment 100 contains an example of an environment for the execution of at least some of the computer code involved in performing the inventive methods, such as predicting, using an artificial intelligence enabled system, an incremental degradation of a battery of a transportation vehicle, calculating an equivalent carbon footprint, calculating an equivalent carbon footprint tax, and assessing the tax against the vehicle. Embodiments of this disclosure can be embodied in computer program product 190. In addition to computer program product 190, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and computer program product 190, as identified above), peripheral device set 114 (including user interface (UI) device set 123, storage 124, and Internet of Things (IoT) sensor set 125), and network module 115.

Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile sequestering device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in computer program product 190 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in computer program product 190 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer, and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

In the illustrative examples, the hardware can take a form selected from at least one of a circuit system, an integrated circuit, an application specific integrated circuit (ASIC), a programmable logic device, or some other suitable type of hardware configured to perform a number of operations. With a programmable logic device, the device can be configured to perform the number of operations. The device can be reconfigured at a later time or can be permanently configured to perform the number of operations. Programmable logic devices include, for example, a programmable logic array, a programmable array logic, a field programmable logic array, a field programmable gate array, and other suitable hardware devices. Additionally, the processes can be implemented in organic components integrated with inorganic components and can be comprised entirely of organic components excluding a human being. For example, the processes can be implemented as circuits in organic semiconductors.

As used herein, “off-gas” and “off-gassing” means airborne release of a chemical or combination of chemicals in vapor form. In addition, as used herein, “off-gas” and “off-gassing” also means release of a chemical or combination of chemicals in gas or liquid form. In addition, as used herein, “off-gas” and “off-gassing” also means release of a gas or chemical that was dissolved, trapped, frozen, or absorbed in a material including sublimation and evaporation, as well as desorption, seepage from cracks or internal volumes, and gaseous products of slow chemical reactions (aka outgassing).

As used herein, “correlating” means determining or establishing a mutual relationship or connection or pattern, in which one thing affects, reacts, or depends on another, whether causal or not. For example, establishing, calculating, and/or measuring a relationship between 2 or more variables, such as determining a linear relationship or curve fitting a non-linear relationship.

As used herein, “a number of” when used with reference to items, means one or more items. For example, “a number of parameters” is one or more parameters. As another example, “a number of operations” is one or more operations.

Further, the phrase “at least one of,” when used with a list of items, means different combinations of one or more of the listed items can be used, and only one of each item in the list may be needed. In other words, “at least one of” means any combination of items and number of items may be used from the list, but not all of the items in the list are required. The item can be a particular object, a thing, or a category.

For example, without limitation, “at least one of item A, item B, or item C” may include item A, item A and item B, or item B. This example also may include item A, item B, and item C, or item B and item C. Of course, any combination of these items can be present. In some illustrative examples, “at least one of” can be, for example, without limitation, two of item A; one of item B; and ten of item C; four of item B and seven of item C; or other suitable combinations.

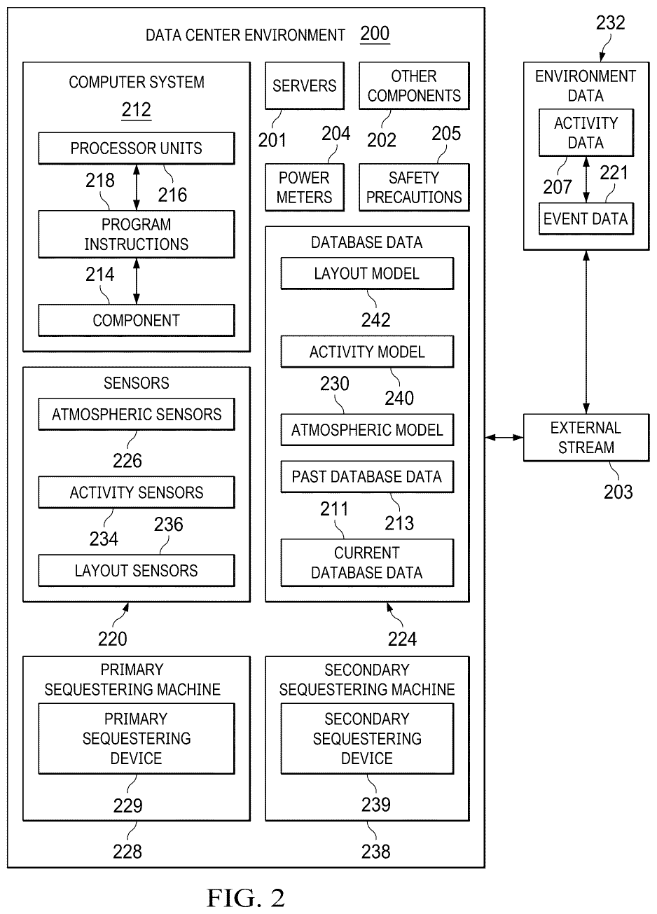

With reference now to FIG. 2, a block diagram of a data center environment is depicted in accordance with an illustrative embodiment. In this illustrative example, data center environment 200 includes components that can be implemented in hardware such as the hardware shown in computing environment 100 in FIG. 1. Of course, all the components within the block diagram representing data center environment 200 can communicate with one another.

Data center environment 200 includes computer system 212. Computer system 212 can be, in some embodiments, a DCIM (data center infrastructure management) computer system. Data center environment 200 includes servers 201 and other components 202 that can emit volatile organic compounds into the data center environment 200 such as switches, patch panels, CRAC units, etc. Servers 201 and other components 202 can include log data for activity and power specific to themselves.

External stream 203 is coupled to data center environment 200 and can provide awareness of and data from outside of data center environment 200. External stream 203 can convey external environment data 232. External environment data 232 can include activity data 207 and event data 221. Activity data 207 is from systems outside data center environment 200. Event data 221 can be information regarding an event outside data center environment 200 such as, for example, construction activity outside data center environment 200 or an external power supply outage having a scope beyond data center environment 200.

Database data 224 can include current database data 211 and/or past database data 213. Computer system 212 includes component 214. Component 214 may be termed an extractor, capturer, creator, correlator, and/or repositioner because component 214 implements a number of these functions such as one, some, or all of these functions. For example, component 214 can be I/O hardware that allows communication between a DCIM computer system and external stream 203. In particular, component 214 may be deployed and/or implemented using computer program product 190 in FIG. 1.

Component 214 can be implemented in software, hardware, firmware or a combination thereof. When software is used, the operations performed by component 214 can be implemented in program instructions configured to run on hardware, such as a processor unit. When firmware is used, the operations performed by component 214 can be implemented in program instructions and data and stored in persistent memory to run on a processor unit. When hardware is employed, the hardware can include circuits that operate to perform the operations in component 214.

Computer system 212 is a physical hardware system and includes one or more data processing systems. When more than one data processing system is present in computer system 212, those data processing systems are in communication with each other using a communications medium. The communications medium can be a network. The data processing systems can be selected from at least one of a computer, a server computer, a tablet computer, or some other suitable data processing system.

As depicted, computer system 212 includes a number of processor units 216 that are capable of executing program instructions 218 implementing processes in the illustrative examples. In other words, program instructions 218 are computer readable program instructions.

As used herein, a processor unit in the number of processor units 216 is a hardware device and is comprised of hardware circuits such as those on an integrated circuit that respond to and process instructions and program code that operate a computer. A processor unit can be implemented using processor set 110 in FIG. 1. When the number of processor units 216 executes program instructions 218 for a process, the number of processor units 216 can be one or more processor units that are in the same computer or in different computers. In other words, the process can be distributed between processor units 216 on the same or different computers in computer system 212.

Further, the number of processor units 216 can be of the same type or different types of processor units. For example, the number of processor units 216 can be selected from at least one of a single core processor, a dual-core processor, a multi-processor core, a general-purpose central processing unit (CPU), a graphics processing unit (GPU), a digital signal processor (DSP), or some other type of processor unit.

Computer system 212 can be configured to perform at least one of the steps, operations, or actions described in the different illustrative examples using software, hardware, firmware or a combination thereof. As a result, computer system 212 operates as a special purpose computer system in which component 214 in computer system 212 enables extracting a physical layout of a data center from a database; extracting an activity level from each of a plurality of systems located in the data center; capturing a set of volatile organic compound data within the data center; creating a volatile organic compound map based on the physical layout and the set of volatile organic compound data; correlating the activity level with the volatile organic compound map; and repositioning a mobile volatile organic compound sequestering device to one or more locations in the data center based on the correlated activity level and volatile organic compound map and the physical layout. In particular, component 214 transforms computer system 212 into a special purpose computer system as compared to currently available general computer systems that do not have component 214. The phrase plurality of systems is intended to be a very broad element; not intended to be limited to a multiplicity of computer systems such as computer system 212. Of note, there can be other systems, such as for example servers, running in the data center.

In the illustrative example, the use of component 214 in computer system 212 integrates processes into a practical application for repositioning a mobile volatile organic compound sequestering device to one or more locations in the data center based on the correlated activity level and volatile organic compound map and the physical layout that increases the sequestering performance of computer system 212. In other words, component 214 in computer system 212 is directed to a practical application of processes integrated into component 214 in computer system 212 that controls repositioning one or more mobile volatile organic compound sequestering device(s) to one or more locations in the data center based on the correlated activity level and volatile organic compound map and the physical layout. In this illustrative example, component 214 in computer system 212 can also control predicting a future volatile organic compound map using an off-gassing decay factor, adjusting a cooling system associated with the new system in the data center to run a new system at a higher temperature than other systems in the data center, capturing airflow data within the data center. Safety precautions 205 can include, at least one of activating an audible alarm, activating a visual alarm, and locking one or more doors of the data center. In this manner, component 214 in computer system 212 provides a practical application of extracting a physical layout of a data center from a database; extracting an activity level from each of a plurality of systems located in the data center; capturing a set of volatile organic compound data within the data center; creating a volatile organic compound map based on the physical layout and the set of volatile organic compound data; correlating the activity level with the volatile organic compound map; and repositioning a mobile volatile organic compound sequestering device to one or more locations in the data center based on the correlated activity level and volatile organic compound map and the physical layout such that the functioning of data center environment 200 is improved. Of note, there can be other systems running in the data center.

Data center environment 200 includes sensors 220. Sensors 220 can be digital or analog. Sensors 220 can include atmospheric sensors 226 such as temperature, humidity, pressure, VOC, gas and/or airflow. Sensors 220 can include activity sensors 234 such as power consumption, workload and/or calculation accumulators. Sensors 220 can include layout sensors 236 such as laser rangefinders and/or ultrasonic detectors. Data from these sensors can be recorded and/or used for extracting a physical layout of a data center from a database; extracting an activity level from each of a plurality of systems located in the data center; capturing a set of volatile organic compound data within the data center; creating a volatile organic compound map based on the physical layout and the set of volatile organic compound data; correlating the activity level with the volatile organic compound map; and repositioning a mobile volatile organic compound sequestering device to one or more locations in the data center based on the correlated activity level and volatile organic compound map and the physical layout, or other purposes. Of note, there can be other systems running in the data center.

The data center environment 200 includes primary sequestering machine 228. Primary sequestering machine 228 can include a primary sequestering device 229. The primary sequestering device can be contained within the sequestering machine and can be a component or sub-component that can be replaced or serviced more easily and less expensively than replacing or servicing the corresponding machine. For example, the primary sequestering device can be a reversibly removable (sub-)component that includes a zeolite and/or activated carbon. Database data 224 can include atmospheric model 230. Atmospheric model 230 can be used to create a volatile organic compound map.

Atmospheric model 230 can include a log of all atmospheric data from atmospheric sensors 226 and any atmospheric data reported by sensors located in the sequestering machines and/or devices, servers 201, and other components 202.

The data center environment 200 can include secondary sequestering machine 238. Secondary sequestering machine 238 can include a secondary sequestering device 239. The secondary sequestering device can be contained within the sequestering machine and can be a component or sub-component that can be replaced or serviced more easily and less expensively than replacing or servicing the corresponding machine. For example, the secondary sequestering device can be a reversibly removable (sub-)component that includes a zeolite and/or activated carbon.

Database data 224 can include activity model 240. Activity model 240 can be used to correlate the activity level with the volatile organic compound map. Activity model 240 can be historical data of all activity/power information extracted from each of the servers 201, other components 202, power meters 204, activity sensors 234, sequestering machines 228 and 239, and sequestering devices 229 and 239.

Database data 224 can include layout model 242. Layout model 242 can be used to create the volatile organic compound map. Layout model 242 can be used to reposition a mobile volatile organic compound sequestering machine and its device. Layout model 242 can be data that describes the physical layout of data center environment 200 and where each item within the environment is physically located as well as the vertical position within the rack which could be extracted from layout sensors 236 or based on a physical log created by users within the data center as systems are installed/removed. This should also include the position of each of the sensors.

The illustration of the data center and its environment in FIG. 2 is not meant to imply physical or architectural limitations to the manner in which an illustrative embodiment can be implemented. Other components in addition to or in place of the ones illustrated may be used. Some components may be unnecessary. Also, the blocks are presented to illustrate some functional components. One or more of these blocks may be combined, divided, or combined and divided into different blocks when implemented in an illustrative embodiment.

In some embodiments, a sequestering machine roams around a data center. In some embodiments, the sequestering machine does not have to roam the entire data center if sensors are located throughout the data center. In other embodiments, the roaming only occurs if the sensors in the data center are not present throughout the data center. In some embodiments, the sequestering machine roams around one or more portions of the data center that are not fully equipped with sensors. In some embodiments, based on all or some sensor data (in the data center and on the sequestering machines and/or sequestering devices) a sequestering machine may be repositioned in an area that the sequestering machine is not roaming regularly or monitoring to sequester VOCs in that location. In some embodiments a sequestering machine follows a user around a data center.

Embodiments of this disclosure can include the following steps.
Step 1. Extract data center device layout.

Could be extracted from computer system 212 or other database.
Step 2. Is a new device (e.g., server) detected?

If yes, position the sequestering machine behind new device that is likely to off-gas high levels of VOCs.

In some embodiments, facility cooling could be adjusted to run the new equipment at a higher ambient such that it off-gasses VOCs faster when the sequestering machine is in position.

In some embodiments, the sequestering machine remains in this position for 24 hours, then loops back to step 1.
Step 3. Extract power/workload of systems within the data center and store to a database.
Step 4. Navigate entire data center to extract VOC sensor readings and create VOC level mapping and store to a database.

In some embodiments, the sequestering machine performs the sensor readings and mapping periodically (e.g., once per day, once per week, or when an unexpected change in power/workload is detected).

In alternate embodiments, airflow/temperature/humidity sensors could be used to monitor airflow within the data center and store airflow mapping to current database data 211 and/or past database data 213.

In some embodiments, these are direct reading VOC sensors/detectors that monitor one or more of formaldehyde, acetaldehyde toluene, etc.

In some embodiments, security risks may exist in certain locations (e.g., attack on a government data center). In these situations, the sensors may detect more harmful toxic gasses (e.g., Chlorine, sarin, VX, etc.)
Step 5. Correlate power/workload data with VOC level mapping.
Step 6. Position sequestering machine in location where VOCs are the highest or where one or more VOCs are at high threshold levels.

In some embodiments, the sequestering machine uses the historical power/workload data to determine its position and uses an off-gas decay factor to predict a new VOC mapping between sensor capturing cycles.

In the alternate embodiments, position the sequestering machine in a location with high airflow where it is likely to capture more VOCs.

In some embodiments, the position of the sequestering machine is included in a notification to system administrators and/or captured in an event log.

In embodiments where more harmful toxic gasses are detected, the sequestering machine may instead be positioned to follow a user that is in the data center. Additional security measures via computer system 212 include locking the doors to the data center, releasing a remedy into atmosphere from the sequestering machine, sending an alert, etc.
Step 7. Loop back to step 1.

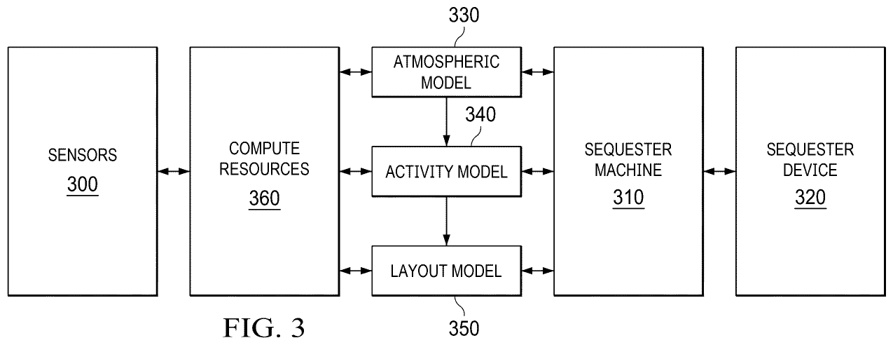

Turning next to FIG. 3, a dataflow diagram of embodiments of this disclosure is depicted. The data in block 360 can be termed compute resources. The data in block 360 interacts with the data in block 330, the data in block 340, and the data in block 350. The data in block 330 can be termed atmospheric model and interacts with the data in block 300. The data in block 340 can be termed activity model and interacts with the data in block 300. The data in block 350 can be termed layout model and interacts with the data in block 300. The data in block 300 can be termed sensors.

The data in block 300 interacts with the data in block 310. The data in block 310 includes instructions from block 360 and can be termed sequester machine. The data in block 310 interacts with the data in block 320. The data in block 320 includes instructions from block 360 and can be termed sequester device.

Sensors represented by block 300 relate to sensors 220 in FIG. 2. Compute resources represented by block 360 relate to computer system to 212 in FIG. 2. An atmospheric model represented by block 330 relates to atmospheric model 230 in FIG. 2. An activity model represented by block 340 relates to activity model 240 in FIG. 2. A layout model represented by block 350 relates to layout model 242 in FIG. 2. A sequestering machine represented by block 310 relates to primary sequestering machine 228 and primary sequestering machine 238 in FIG. 2. A sequestering device represented by block 320 relates to primary sequestering device 229 and secondary sequestering device 239 in FIG. 2.

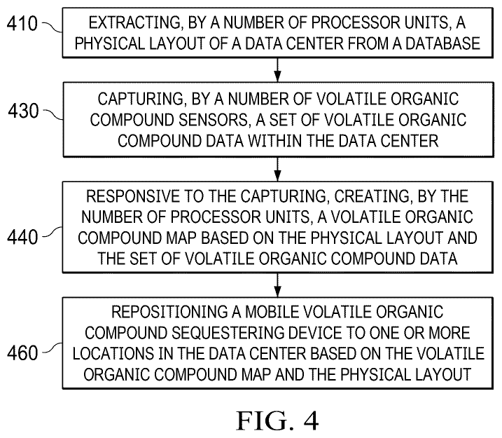

Turning next to FIG. 4, a flowchart of a process for intelligent volatile organic compound capturing in a data center is depicted in accordance with an illustrative embodiment. Embodiments are not limited to the sequence of steps shown in FIG. 4. The process in FIG. 4 can be implemented in hardware, software, or both. When implemented in software, the process can take the form of program instructions that are run by one of more processor units located in one or more hardware devices in one or more computer systems. For example, the process of FIG. 4 can be implemented in component 214 in computer system 212 in FIG. 2.

Block 410 extracts, by a number of processor units, a physical layout of a data center from a database. The physical layout could come from layout model 242 in FIG. 2.

Block 430 captures, by a number of volatile organic compound sensors, a set of volatile organic compound data within the data center. Block 440 responsive to the capturing, creates, by the number of processor units, a volatile organic compound map based on the physical layout and the set of volatile organic compound data. Block 460 repositions a mobile volatile organic compound sequestering device to one or more locations in the data center based on the volatile organic compound map and the physical layout. As shown in FIG. 2, the sequestering device is located within the sequestering machine. The mobile volatile organic compound sequestering machine that includes the mobile volatile organic compound sequestering device can be driven by wheels or other locomotive mechanisms.

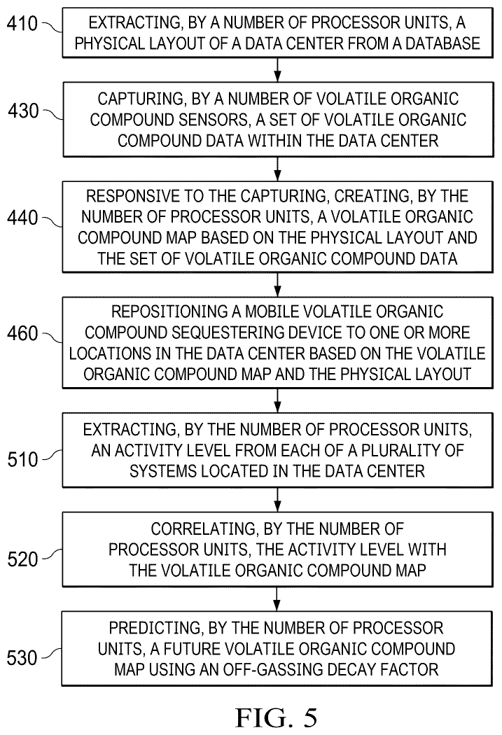

Turning now to FIG. 5, a flowchart depicts optional additional steps that can be combined with the steps of FIG. 4 in accordance with an illustrative embodiment. Embodiments are not limited to the sequence of steps shown in FIG. 5. The process in FIG. 5 can be implemented in hardware, software, or both. When implemented in software, this process can take the form of program instructions that are run by one of more processor units located in one or more hardware devices in one or more computer systems. For example, the process of FIG. 5 can be implemented in component 214 in computer system 212 in FIG. 2. The steps from FIG. 4 are repeated in FIG. 5 and can be the same steps. Of course, embodiments are not limited to the sequence of steps shown in FIG. 4 and/or FIG. 5. For example, in a different embodiment blocks 410, 430 and 440 could hypothetically precede blocks 510, 520 and 530 with block 460 following block 530 instead of block 460 preceding blocks 510, 520 and 530. Block 510 describes extracting, by the number of processor units, an activity level from each of a plurality of systems located within the data center. Block 520 describes correlating, by the number of processor units, the activity level with the volatile organic compound map. Block 530 describes predicting, by the number of processor units, a future volatile organic compound map using an off-gassing decay factor. Predicting a future volatile organic compound map using an off-gassing decay factor could be based on predicted future patterns in system activity. Alternatively, trends in VOC off-gassing with a decay factor may emerge based on utilization/activity without having to specifically monitor that activity and correlate it to the measured VOCs. Of course, embodiments are not limited to the sequence of steps shown in FIG. 4 and/or FIG. 5 and embodiments are open to other functions such as decision blocks as well as being open to additional step(s). Of note, there can be other systems running in the data center.

In an embodiment, activity level includes at least one of power consumption and workload from each of a plurality of systems located in the data center. Of note, there can be other systems running in the data center.

In an embodiment, capturing the set of volatile organic compound data within the data center is performed at regular intervals.

In an embodiment, capturing is performed responsively to detecting an unexpected change in activity level on one or more systems in the datacenter.

In an embodiment, repositioning includes repositioning to at least one of where a volatile organic compound is highest and where a volatile organic compound exceeds a threshold level.

In an embodiment, capturing is performed responsive to detecting an unexpected change in activity level (e.g. power and/or workload) on one or more systems in the datacenter. As a result, these illustrative embodiments provide a technical effect of sequestering volatile organic compounds responsive to detecting an unexpected change in activity level on one or more systems in the datacenter.

In an embodiment, repositioning comprises repositioning to at least one of where a volatile organic compound is highest and where a volatile organic compound exceeds a threshold level. For example, highest above its threshold or highest as a percentage of its threshold (e.g., a first VOC may be 200% of its threshold and have a lower threshold than a second VOC which is 130% of its threshold. If the threshold for the second VOC is very high, the overall levels at the 130% reading may be higher than the overall levels of the first VOC at the 200% reading. Because the first VOC is more hazardous and further above its threshold, embodiments can reposition there even though the overall levels are lower than the second VOC).

In an embodiment, repositioning can include following a user moving within the data center.

In an embodiment, repositioning can be based on at least one of historical power consumption and historical workload.

An embodiment can include predicting a future volatile organic compound map using an off-gassing decay factor before repeating capturing, by the number of volatile organic compound sensors, the set of volatile organic compound data within the data center.

An embodiment can include, responsive to detecting a new system in the data center, repositioning the mobile volatile organic compound sequestering device approximate the new system.

An embodiment can include responsive to repositioning the mobile volatile organic compound sequestering device proximate a new system, adjusting a dedicated cooling system associated with the new system in the data center to run the new system at a higher temperature than a plurality of systems to increase off-gassing of the volatile organic compounds from the new system. Of note, there can be other systems running in the data center; the dedicated cooling system associated with the new system is not to be confused with other cooling systems regardless of whether the dedicated association is one or more of permanent, transient, and intermittent. Of note, there can be other systems running in the data center.

An embodiment can include capturing air flow within the data center.

An embodiment can include, if a threshold is exceeded, at least one of activating an audible alarm, activating a visual alarm, and locking one or more doors of the data center.

The flowcharts and block diagrams in the different depicted embodiments illustrate the architecture, functionality, and operation of some possible implementations of apparatuses and methods in an illustrative embodiment. In this regard, each block in the flowcharts or block diagrams may represent at least one of a module, a segment, a function, or a portion of an operation or step. For example, one or more of the blocks can be implemented as program instructions, hardware, or a combination of the program instructions and hardware. When implemented in hardware, the hardware may, for example, take the form of integrated circuits that are manufactured or configured to perform one or more operations in the flowcharts or block diagrams. When implemented as a combination of program instructions and hardware, the implementation may take the form of firmware. Each block in the flowcharts or the block diagrams can be implemented using special purpose hardware systems that perform the different operations or combinations of special purpose hardware and program instructions run by the special purpose hardware.

In some alternative implementations of an illustrative embodiment, the function or functions noted in the blocks may occur out of the order noted in the figures. For example, in some cases, two blocks shown in succession can be performed substantially concurrently, or the blocks may sometimes be performed in the reverse order, depending upon the functionality involved. Also, other blocks can be added in addition to the illustrated blocks in a flowchart or block diagram.

A practical application of an embodiment of the present disclosure that has value within the technological arts is a hypothetical situation where a threat actor releases one or more types of VOCs into a data center to achieve one or more of the following objectives. First, immediately disrupt, contaminate, or interfere with the active processing of IT resources, environmental systems (e.g., heating, cooling, conditioning), and/or security systems (e.g., card readers, emissions sensors, etc.). Second, create an environment that harms humans. Third, over time, alter the operating “air” in order to lead to transient micro-outages, accelerated degradation of IT parts (thus increasing total cost of ownership and/or leading to more outages), harm staff, etcetera. A mobile volatile organic compound sequestering device can be instrumented to detect VOCs that could cause one or both of the first and second objectives. A secondary benefit is that data collected over time can be analyzed as an indicator that the third objective may be vulnerable to completion by the threat actor. There are virtually innumerable uses for embodiments of the present disclosure, all of which need not be detailed here.

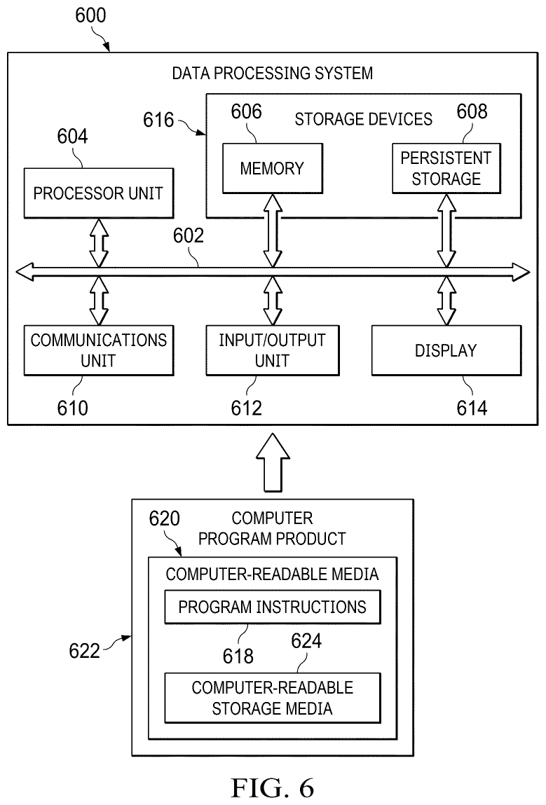

Turning now to FIG. 6, a block diagram of a data processing system is depicted in accordance with an illustrative embodiment. Data processing system 600 can be used to implement computers and computing devices in computing environment 100 in FIG. 1. Data processing system 600 can also be used to implement computer system 212 in FIG. 2. In this illustrative example, data processing system 600 includes communications framework 602, which provides communications between processor unit 604, memory 606, persistent storage 608, communications unit 610, input/output (I/O) unit 612, and display 614. In this example, communications framework 602 takes the form of a bus system.

Processor unit 604 serves to execute instructions for software that can be loaded into memory 606. Processor unit 604 includes one or more processors. For example, processor unit 604 can be selected from at least one of a multicore processor, a central processing unit (CPU), a graphics processing unit (GPU), a physics processing unit (PPU), a digital signal processor (DSP), a network processor, or some other suitable type of processor. Further, processor unit 604 can be implemented using one or more heterogeneous processor systems in which a main processor is present with secondary processors on a single chip. As another illustrative example, processor unit 604 can be a symmetric multi-processor system containing multiple processors of the same type on a single chip.

Memory 606 and persistent storage 608 are examples of storage devices 616. A storage device is any piece of hardware that is capable of storing information, such as, for example, without limitation, at least one of data, program instructions in functional form, or other suitable information either on a temporary basis, a permanent basis, or both on a temporary basis and a permanent basis. Storage devices 616 may also be referred to as computer readable storage devices in these illustrative examples. Memory 606, in these examples, can be, for example, a random-access memory or any other suitable volatile or non-volatile storage device. Persistent storage 608 may take various forms, depending on the particular implementation.

For example, persistent storage 608 may contain one or more components or devices. For example, persistent storage 608 can be a hard drive, a solid-state drive (SSD), a flash memory, a rewritable optical disk, a rewritable magnetic tape, or some combination of the above. The media used by persistent storage 608 also can be removable. For example, a removable hard drive can be used for persistent storage 608.

Communications unit 610, in these illustrative examples, provides for communications with other data processing systems or devices. In these illustrative examples, communications unit 610 is a network interface card.

Input/output unit 612 allows for input and output of data with other devices that can be connected to data processing system 600. For example, input/output unit 612 may provide a connection for user input through at least one of a keyboard, a mouse, or some other suitable input device. Further, input/output unit 612 may send output to a printer. Display 614 provides a mechanism to display information to a user.

Instructions for at least one of the operating system, applications, or programs can be located in storage devices 616, which are in communication with processor unit 604 through communications framework 602. The processes of the different embodiments can be performed by processor unit 604 using computer-implemented instructions, which may be located in a memory, such as memory 606.

These instructions are referred to as program instructions, computer usable program instructions, or computer readable program instructions that can be read and executed by a processor in processor unit 604. The program instructions in the different embodiments can be embodied on different physical or computer readable storage media, such as memory 606 or persistent storage 608.

Program instructions 618 are located in a functional form on computer readable media 620 that is selectively removable and can be loaded onto or transferred to data processing system 600 for execution by processor unit 604. Program instructions 618 and computer readable media 620 form computer program product 622 in these illustrative examples. In the illustrative example, computer readable media 620 is computer readable storage media 624.

Computer readable storage media 624 is a physical or tangible storage device used to store program instructions 618 rather than a medium that propagates or transmits program instructions 618. Computer readable storage media 624, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Alternatively, program instructions 618 can be transferred to data processing system 600 using a computer readable signal media. The computer readable signal media are signals and can be, for example, a propagated data signal containing program instructions 618. For example, the computer readable signal media can be at least one of an electromagnetic signal, an optical signal, or any other suitable type of signal. These signals can be transmitted over connections, such as wireless connections, optical fiber cable, coaxial cable, a wire, or any other suitable type of connection.

Further, as used herein, “computer readable media 620 can be singular or plural. For example, program instructions 618 can be located in computer readable media 620 in the form of a single storage device or system. In another example, program instructions 618 can be located in computer readable media 620 that is distributed in multiple data processing systems. In other words, some instructions in program instructions 618 can be located in one data processing system while other instructions in program instructions 618 can be located in one data processing system. For example, a portion of program instructions 618 can be located in computer readable media 620 in a server computer while another portion of program instructions 618 can be located in computer readable media 620 located in a set of client computers.

The different components illustrated for data processing system 600 are not meant to provide architectural limitations to the manner in which different embodiments can be implemented. In some illustrative examples, one or more of the components may be incorporated in or otherwise form a portion of, another component. For example, memory 606, or portions thereof, may be incorporated in processor unit 604 in some illustrative examples. The different illustrative embodiments can be implemented in a data processing system including components in addition to or in place of those illustrated for data processing system 600. Other components shown in FIG. 6 can be varied from the illustrative examples shown. The different embodiments can be implemented using any hardware device or system capable of running program instructions 618.

Thus, illustrative embodiments of the present disclosure provide a computer-implemented method, computer system, and computer program product for repositioning a sequestering device in a data center. The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer implemented method comprising:
extracting, by a number of processor units, a physical layout of a data center from a database;
capturing, by a number of volatile organic compound sensors, a set of volatile organic compound data within the data center;
responsive to the capturing, creating, by the number of processor units, a volatile organic compound map based on the physical layout and the set of volatile organic compound data; and
repositioning a mobile volatile organic compound sequestering device to one or more locations in the data center based on the volatile organic compound map and the physical layout.

2. The computer implemented method of claim 1, wherein capturing, by the number of volatile organic compound sensors, the set of volatile organic compound data within the data center is performed at regular intervals.

3. The computer implemented method of claim 1, wherein capturing is performed responsive to detecting an unexpected change in activity level on one or more systems in the data center.

4. The computer implemented method of claim 1, wherein repositioning comprises repositioning to at least one of where a volatile organic compound is highest above its threshold or highest as a percentage of its threshold.

5. The computer implemented method of claim 1, wherein repositioning comprises following a user moving within the data center.

6. The computer implemented method of claim 1, wherein repositioning is based on at least one of historical power consumption and historical workload.

7. The computer implemented method of claim 1, further comprising
extracting, by the number of processor units, an activity level from each of a plurality of systems located in the data center;
correlating, by the number of processor units, the activity level with the volatile organic compound map; and
predicting, by the number of processor units, a future volatile organic compound map using an off-gassing decay factor.

8. The computer implemented method of claim 7, wherein the activity level includes at least one of power consumption and workload from each of the plurality of systems located in the data center.

9. The computer implemented method of claim 1, further comprising, responsive to detecting a new system in the data center, repositioning the mobile volatile organic compound sequestering device proximate the new system.

10. The computer implemented method of claim 1, further comprising, responsive to repositioning the mobile volatile organic compound sequestering device proximate a new system, adjusting a cooling system associated with the new system in the data center to run the new system at a higher temperature than a plurality of systems to increase off-gassing of volatile organic compounds from the new system.

11. The computer implemented method of claim 1, further comprising capturing, by a number of airflow sensors, airflow data within the data center.

12. The computer implemented method of claim 1, further comprising, responsive to a volatile organic compound threshold being exceeded, at least one of activating an audible alarm, activating a visual alarm, and locking one or more doors of the data center.

13. A computer implemented method comprising:
extracting, by a number of processor units, a physical layout of a data center from a data center infrastructure management tool;
capturing, by a number of toxic gas sensors, a set of toxic gas data within the data center; responsive to the capturing, creating, by the number of processor units, a toxic gas map based on the physical layout and the set of toxic gas data; and
repositioning a mobile toxic gas sequestering device in one or more locations in the data center based on the toxic gas map and the physical layout.

14. The computer implemented method of claim 13, further comprising
extracting, by the number of processor units, an activity level from each of a plurality of systems located in the data center; and
correlating, by the number of processor units, the activity level with the toxic gas map.

15. The computer implemented method of claim 13, further comprising capturing, by a number of airflow sensors, airflow data within the data center.

16. The computer implemented method of claim 13, further comprising, responsive to a toxic gas threshold being exceeded, at least one of activating an audible alarm, activating a visual alarm, and locking one or more doors of the data center.

17. A computer implemented method comprising:
extracting, by a number of processor units, a physical layout of a data center from a data center infrastructure management tool;
capturing, by a number of airflow sensors, a set of airflow data within the data center;
responsive to the capturing, creating, by the number of processor units, an airflow map based the physical layout and the set of airflow data; and
repositioning a mobile sequestering device to one or more locations in the data center based on the airflow map and the physical layout.

18. The computer implemented method of claim 17, further comprising, responsive to repositioning the mobile sequestering device proximate a new system, adjusting a cooling system associated with the new system in the data center to run the new system at a higher temperature than a plurality of systems to increase off-gassing of volatile organic compounds from the new system.

19. The computer implemented method of claim 17, further comprising capturing, by a number of airflow sensors, airflow data within the data center.

20. The computer implemented method of claim 17, further comprising
extracting, by the number of processor units, an activity level from each of a plurality of systems located in the data center; and
correlating, by the number of processor units, the activity level with the airflow map.

21. A computer system comprising:
a processor set;
a set of one or more computer readable storage media;
program instructions, collectively stored in the set of one or more storage media, for causing the processor set to perform the following computer operations:
extract, by a number of processor units, a physical layout of a data center from a database;
capture, by a number of volatile organic compound sensors, a set of volatile organic compound data within the data center;
responsive to the capture, create, by the number of processor units, a volatile organic compound map based on the physical layout and the set of volatile organic compound data; and
reposition a mobile volatile organic compound sequestering device to one or more locations in the data center based on the volatile organic compound map and the physical layout.

22. The computer system of claim 21, wherein the program instructions cause the processor set to perform the following computer operations:
extract, by the number of processor units, an activity level from each of a plurality of systems located in the data center; and
correlate, by the number of processor units, the activity level with the volatile organic compound map.

23. The computer system of claim 21, wherein the program instructions cause the processor set to perform the following computer operations:
capture, by a number of airflow sensors, airflow data within the data center.

24. The computer system of claim 21, wherein the program instructions cause the processor set to perform at least one of the following computer operations:
responsive to a volatile organic compound threshold being exceeded, activate an audible alarm, activate a visual alarm, and lock one or more doors of the data center.

25. A computer program product comprising:
a set of one or more computer-readable storage media;
program instructions, collectively stored in the set of one or more storage media, for causing a processor set to perform the following computer operations:
extract, by a number of processor units, a physical layout of a data center from a database;
capture, by a number of volatile organic compound sensors, a set of volatile organic compound data within the data center;
responsive to the capture, create, by the number of processor units, a volatile organic compound map based on the physical layout and the set of volatile organic compound data; and
reposition a mobile volatile organic compound sequestering device to one or more locations in the data center based on the volatile organic compound map and the physical layout.
